(19) United States
(12) Patent Application Publication
(10) Pub. No.: US 2020/0339582 A1
(43) Pub. Date: Oct. 29, 2020

(54) Atypical Carbapenem Antibiotics with Improved Activity Against Carbapenemase-Producing Acinetobacter baumannii

(71) Applicants: John Buynak, Dallas, TX (US); Sergei Vakulenko, Granger, IN (US); Maha Alqurafi, Dallas, TX (US); Noora Al-Kharji, Doha (QA); Marta Toth, Granger, IN (US); Nichole Stewart, South Bend, IN (US)

(72) Inventors: John Buynak, Dallas, TX (US); Sergei Vakulenko, Granger, IN (US); Maha Alqurafi, Dallas, TX (US); Noora Al-Kharji, Doha (QA); Marta Toth, Granger, IN (US); Nichole Stewart, South Bend, IN (US)

(21) Appl. No.: 16/839,049

(22) Filed: Apr. 2, 2020

Related U.S. Application Data
(60) Provisional application No. 62/828,436, filed on Apr. 2, 2019.

(51) Int. Cl. C07D 477/26

(52) U.S. Cl. CPC C07D 477/26

(57) ABSTRACT

The following invention deals with the design, preparation, evaluation, and use of carbapenem antibiotics with improved activity, relative to current commercially available carbapenem antibiotics, against infections involving multidrug resistant, carbapenemase-producing Acinetobacter baumannii. The new carbapenem antibiotics are demonstrated to possess not only inherent antimicrobial activity, but also the ability to inhibit OXA-23, the most commonly produced serine carbapenemase in this species. This unusual carbapenemase-inhibitory activity also indicates that the compounds may be used synergistically, in combination with current commercial carbapenem antibiotics, to inhibit key class D carbapenemases, such as OXA-23. Additionally, one of the newly reported carbapenems is active against metallo-beta-lactamase producing A. baumannii. This is the first report of a metallo-beta-lactamase stable carbapenem antibiotic. Structurally, the present invention describes carbapenem antibiotics which are modified in unusual ways, thus differentiating them from the common scaffold of all current commercial carbapenem antibiotics. In particular, these carbapenems have either an unusual C6 substituent, a hydroxymethyl group, replacing the common hydroxyethyl group, or they have an unusual C5 substituent, an alkyl group, replacing the common hydrogen atom at this position. Such atypical carbapenem antibiotics have not previously been investigated against resistant A. baumannii, nor have they been evaluated for stability to the class D carbapenemase, or the class B metallo-beta-lactamases.

The present applications claims priority to the earlier filed provisional application having Ser. No. 62/828,436, and hereby incorporates subject matter of the provisional application in its entirety.

### STATEMENT REGARDING FEDERALLY SPONSORED RESEARCH

This research was supported through grants from the National Institutes of Health (A1109624 and A1142699 to JDB and A1089726 to SV)

### CROSS REFERENCE TO RELATED APPLICATIONS

(Not Applicable)

### THE NAMES OF THE PARTIES TO A JOINT RESEARCH AGREEMENT

(Not Applicable)

### INCORPORATION-BY-REFERENCE OF MATERIAL SUBMITTED ON A COMPACT DISK

(Not Applicable)

### BRIEF DESCRIPTION OF THE SEVERAL VIEWS OF THE DRAWINGS

FIG. 1: General structures of atypical carbapenem antibiotics A and B Scheme 1: Synthetic procedure leading to carbapenem antibiotic 1 of general structure A Scheme 2: Synthetic procedure leading to carbapenem antibiotics 2a and 2b of general structure B

FIG. 2: General Structures of claims.

### BACKGROUND

The 21st century has seen Acinetobacter baumannii (Ab) evolve into a major bacterial pathogen, primarily due to a dramatic increase in beta-lactamase mediated resistance to the carbapenem antibiotics, widely regarded as last resort agents. In some countries, the incidence of carbapenem resistant Ab (CRAb) has reached 90%. Although Ab can produce all four molecular classes of beta-lactamases (A, C and D of serine- and class B of metallo-enzymes), production of carbapenem-hydrolyzing class D β-lactamases (CHDLs) constitutes, by far, the major mechanism of carbapenem resistance in this pathogen. While Ab produces several common CHDLs (OXA-23, OXA-24/40, OXA-51, OX-58 and OXA-143), OXA-23 is the most prevalent. In addition to production of CHDLs, carbapenem resistance can be caused by mutation of their targets, penicillin-binding proteins (PBPs). Mutations of porins and/or overexpression of efflux pumps, which reduce the concentration of drugs in the cell, can also contribute. Relatively little is known about PBP-, porin- and efflux pump-mediated resistance mechanisms in Ab. CHDL-producing Ab isolates are commonly resistant to multiple or all antibiotics. Consequently, therapeutic options for treatment of Ab infections are limited, translating into high mortality rates, often exceeding 50%. Due to its clinical importance and growing resistance, Ab is included in the list of six bacterial pathogens responsible for the majority of healthcare-associated infections, and is listed by the CDC as a bacterium that poses a serious resistance threat in the U. S.

### DETAILED DESCRIPTION

Carbapenem-resistant A. baumannii most commonly produces the OXA-23 carbapenemase, which is capable of hydrolyzing all commercial carbapenem antibiotics. A carbapenem antibiotic which is resistant to OXA-23-catalyzed hydrolysis would, therefore, be extremely useful in treatment of infections involving this pathogen. The currently described carbapenem antibiotics are not only resistant to hydrolysis, but will also inhibit the OXA-23 carbapenemase, thus providing an opportunity to couple these new carbapenems with other b-lactam antibiotics, including, but not limited to, current commercial carbapenems, to attain enhanced potency in treatment of resistant A. baumannii.

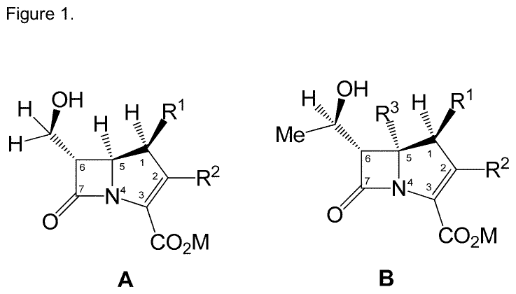

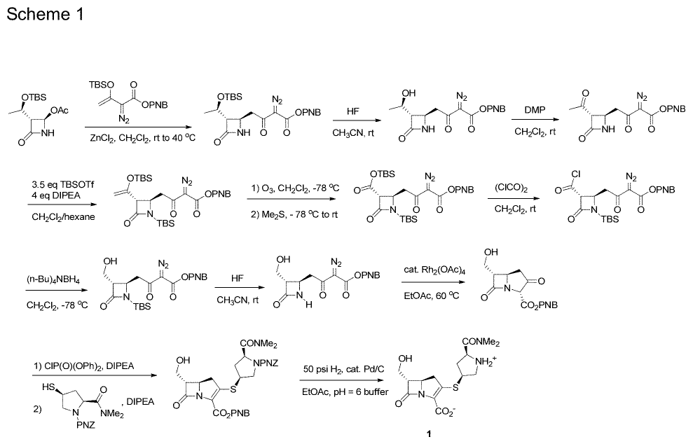

The General Structures of these carbapenems are shown in structures A and B as shown in FIG. 1.

### FIG. 1

Wherein:

Where R¹═H or CH₃

Where R² may be SR^{a}, where R^{a}=may be an unsubstituted C1 to C6 alkyl group, or substituted C1 to C6 alkyl group, especially including substituents which themselves possess a basic nitrogen, and hence a positive charge. Or alternatively R^{a} may be a substituted or unsubstituted, cyclic or heterocylic group, especially including groups which contain 1 to 3 positive charges, an aryl or heteroaryl group, or substituted aryl or heteroaryl group, particularly including a substituted pyrrolidine.

Where R³ may be Methyl or Ethyl.

With respect to —CO2M, which is attached to the carbapenem nucleus at position 3, this represents a carboxylic acid group (M represents H), a carboxylate anion (M represents a negative charge), a pharmaceutically acceptable ester (M represents an ester forming group) or a carboxylic acid protected by a 30 protecting group (M represents a carboxyl protecting group).

The pharmaceutically acceptable salts referred to above may take the form —COOM, where M is a negative charge, which is balanced by a counterion, e.g., an alkali metal cation such as sodium or potassium. Other pharmaceutically acceptable counterions may be calcium, magnesium, zinc, ammonium, or alkylammonium cations such as tetramethylammonium, tetrabutylammonium, choline, triethylhydroammonium, meglumine, triethanolhydroammonium, etc.

The pharmaceutically acceptable salts referred to above also include acid addition salts. Thus, the Formula I compounds can be used in the form of salts derived from inorganic or organic acids. Included among such salts are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate and undecanoate.

The pharmaceutically acceptable esters are such as would be readily apparent to a medicinal chemist, and include, for example, those described in detail in U.S. Pat. No. 4,309,438. Included within such pharmaceutically acceptable esters are those which are hydrolyzed under physiological conditions, such as pivaloyloxymethyl, acetoxymethyl, phthalidyl, indanyl and methoxymethyl, and others described in detail in U.S. Pat. No. 25 4,479,947. These are also referred to as “biolabile esters”.

Biolabile esters are biologically hydrolizable, and may be suitable for oral administration, due to good absorption through the stomach or intestinal mucosa, resistance to gastric acid degradation and other factors. Examples of biolabile esters include compounds in which M represents an alkoxyalkyl, alkylcarbonyloxyalkyl, alkoxycarbonyloxyalkyl, cycloalkoxyalkyl, alkenyloxyalkyl, aryloxyalkyl, alkoxyaryl, alkylthioalkyl, cycloalkylthioalkyl, alkenylthioalkyl, arylthioalkyl or alkylthioaryl group. These groups can be substituted in the alkyl or aryl portions thereof with acyl or halo groups. The following M species are examples of biolabile ester forming moieties: acetoxymethyl, 1-acetoxyethyl, 1-acetoxypropyl, pivaloyloxymethyl, 1-isopropyloxycarbonyloxyethyl, 1-cyclohexyloxycarbonyloxyethyl, phthalidyl and (2-oxo-5-methyl-1,3-dioxolen-4-yl)methyl.

The synthetic methodology which has been employed to make these new carbapenems is shown in Schemes 1 (example 1 of general structure A) and 2 (examples 2a and 2b of general structure B).

### Scheme 1

### Scheme 2

### Experimental Section, Scheme 1

### p-Nitrobenzyl Acetoacetate

Methyl acetoacetate (118.7 ml, 1.1 mol) and p-nitrobenzyl alcohol (153.1 g, 1 mol), were placed in a 3 L round bottom flask with a distilled collector. The reaction mixture was heated to 190° C., the methanol was allowed to distill, and the reaction was checked (monitoring) using 1H NMR. After completion of the reaction, the mixture was placed under high vacuum to remove the excess methyl acetoacetate. Yield was (249 g, 90%). 1H NMR (400 MHz, CDCl3): delta 8.26 (d, 2H), 7.58 (d, 2H), 5.33 (d, 2H), 3.64 (s, 2H), 2.33 (s, 3H).

### p-nitrobenzyl alpha-diazoacetate

p-Nitrobenzyl acetoacetate (1.1 kg, 4.38 mol) dissolved in acetone (1.54 L) and water (1.54 L) and then cooled to −20° C., and a solution of NaHCO₃(179.2 g, mol) in 1.536 L of water was added. NaN3 (322 g, 4.95 mol) and dimethyl sulfonyl chloride (360 mL, 2.52 mol) were added to the solution slowly subsequently. After addition, the reaction was allowed to warm between 7-8° C. After the reaction completed, acetone was removed under reduced pressure, the residue was diluted with DCM and the organic layer was extracted and dried over NaSO4. Yield was (948 g, 82%), 1H NMR (400 MHz, CDCl3): delta 8.25 (d, 2H), 7.53 (d, 2H), 5.36 (d, 2H), 2.49 (s, 2H).

### 4-nitrobenzyl-2-diazo-3-tert-butyldimethylsilyloxy-3-butenoate

In the ice bath, tert-butyl dimethylsilyl trifluoromethylsulfonate (991 mL, 3.22 mol) was added portionwise to a suspension of p-nitrobenzyl alpha-diazoacetate (948 g, 3.60 mol) and triethylamine(721 mL, 5.02 mol) in dry DCM (2.8 L) under nitrogen atmosphere. The reaction mixture was warmed to 2° C. for one hour. Color of the reaction solution turned from yellow to orange. Brine solution was poured to the reaction mixture, and then organic layer was separated, dried over Na2SO4 and evaporated, yielding (1.21 kg, 89%). 1HNMR (CDCl3, EM-360A, 60 MHz): delta 8.22 (d, 2H), 7.48 (d, 2H), 5.32 (s, 2H), 4.97 (d, 1H), 4.25 (d, 1H), 0.96 (s, 9H), 0.26 (d, 6H). IR (KBr, cm−1) 2090, 1694, 1600, and 1344.

### (p-Nitrophenyl)methyl 4-[3-(1-tertbutyldimethysilyoxyethyl)-4-oxo-2-azetidinyl]-2-diazoacetoacetate

In a 3 neck round bottom flask equipped with overhead stirrer and equipped with N2 balloon, zinc chloride (138 g, 1.01 mole) was added to a solution of 1′R,3R,4R)-3-(1′-tert-butyldimethylsilyloxyethyl)-4-acetoxyazetidin-2-one 1 (389 g, 1.49 mole) in methylene chloride (2 L) followed by solid 4-nitrobenzyl-2-diazo-3-tert-butyldimethylsilyloxy-3-butenoate (562 g, 1.49 mole). The reaction was warmed to 40° C. The mixture was stirred at room temperature under nitrogen for 3 hours. The mixture was washed with saturated sodium bicarbonate (2×25 mL) and then brine, dried (Na2SO4) and evaporated, yielding a crude oily yellow solid. Yield was (392 g, 53%). 1H NMR (400 MHz, CDCl3): delta 8.27 (d, 2H), 7.55 (d, 2H), 6.15 (s, 1H), 5.38 (s, 2H), 4.08 (td, 1H), 3.67 (d, 1H), 3.48 (dd, 1H), 2.86 (dd, 1H), 2.07 (d, 3H), 0.86 (s, 9H), 0.04 (d, 6H).

### (p-Nitrophenyl)methyl 4-[3-(1-hydroxyethyl)-4-oxo-2-azetidinyl]-2-(imino)acetoacetate

30 mL of HF was added to a solution of (p-nitrophenyl)methyl 4-[3-(1-tertbutyldimethysilyoxyethyl)-4-oxo-2-azetidinyl]-2-diazoacetoacetate (392 g, 776 mmol) in 600 mL of CH3CN at rt overnight. The reaction was monitored by thin layer chromatography. Additional 20 mL of HF was added to the reaction to complete. After the reaction completed, fine powder of NaHCO3 was added to the mixture to adjust pH to 8. The solid was filtered and the filtrate was concentrated under reduced pressure to afford a white solid (296 g, 98%). 1H NMR (400 MHz, CDCl3): delta 8.28 (d, 2H), 7.56 (d, 2H), 6.27 (s, 1H), 5.38 (d, 2H), 4.40 (m, 1H), 3.97 (d, 2H), 3.97 (s, 1H), 3.42 (dd, 1H), 3.09 (dd, 1H), 2.34 (s, 3H). 13C NMR (CDCl3, 400 MHz): delta 190, 161, 148, 142, 129, 124, 65.6, 65.1. IR (KBr, cm-1): 3390, 2967, 2142, 1721, 1651, 1608, 1522, 1386, 1347, 1295, 1217, 1129, 1015, 853, 739.

### (p-Nitrophenyl)methyl 4-(3-acetyl-4-oxo-2-azetidinyl)-2-(imino)acetoacetate 31

Method A

Compound 30 (20 g, 53.1 mmol) was dissolved in dry DCM (200 mL). DMP (22.5 g, 55.2 mmol) was added slowly for 20 min. The reaction stirred for 15 min at rt. A solution of sodium thiosulfate pentahydrate (20 g) in a saturated solution of NaHCO₃(300 mL) was added to the reaction and stirred for an additional hour till DMP disappeared in NMR spectra. The organic layer was separated and washed with water. Then, the organic layer was concentrated under reduced pressure. The yield was (18.8 g, 95%). 1H NMR (400 MHz, CDCl3): 1H NMR (400 MHz, CDCl3): delta 8.27 (d, 2H), 7.55 (d, 2H), 6.27 (s, 1H), 5.38 (s, 2H), 4.39 (m, 1H), 3.96 (d, 1H), 3.42 (dd, 1H), 3.11 (dd, 1H), 2.35 (s, 3H). 13C NMR (CDCl3, 400 MHz): delta 189, 163, 161, 142, 129, 124, 68.4, 65.6, 53.4, 45.6, 44.2, 29.7. IR (KBr, cm−1): 3287, 2146, 1763, 1709, 1644, 1523, 1384, 1348, 1295, 1212, 1119, 1014, 853, 696, 718.

Method B

2-compound (2R,3S)-2-(phenylmethyl-2-diazoacetoacetate)-3-[1-hydroxyethyl]azetidin-4-one 30 (251 g, 667 mmol) dissolved in acetone (750 mL) and cooled to 0° C. Jones Reagent [Cr03 (18.4 g), H2SO4 (16.3 mL), water (72 mL)] was added dropwise through additional funnel. The reaction was warmed up to rt and stirred for one hour. The mixture was diluted with EtOAc (1000 mL), and then washed with saturated Na2S2O5 solution till the green color disappeared. The organic layer was separated, dried over sodium sulfate and solvents removed to dryness under reduced pressure. Yield was 78%,

### (p-Nitrophenyl)methyl 4-[3-(1-tertbutyldimethysilyoxyethenyl)-4-oxo-2-azetidinyl]-2-(imino)acetoacetate

(2R,3S)-3-acetyl-2-(phenylmethyl-2-diazoacetoacetate)azetidin-4-one (18.8 g, 38.5 mmol) dissolved in CH2Cl2 (120 mL) first then hexanes (80 mL) added. The reaction was allowed to dissolve and stirred at rt for 5 min. Then the reaction was cooled to −20° C. DIPEA (26.8 mL, 154 mmol) was added followed by TBS-0Tf (35.4 mL, 154 mmol). The reaction was stirred for 40 min at rt. After completion, the resulting mixture was diluted with CH2Cl2 (50 mL), then wash with NaHCO3 and water. Then the organic phase was separated and dried over Na2SO4, concentrated under reduced pressure. The title compound purified by column chromatography eluted (0-10%) DCM: ethyl acetate to obtain (15.7 g, 61%) of the title compound as an oily solid. 1H NMR (400 MHz, CDCl3): delta 8.28 (d, 2H), 7.55 (d, 2H), 5.38 (s, 2H), 4.18 (d, 2H), 3.91 (d, 1H), 3.50 (m, 1H), 2.80 (d, 1H), 2.05 (s, 3H), 1.98 (s, 3H), 1.70 (s, 3H), 0.87 (s, 3H), 0.06 (s, 12H). 13C NMR (400 MHz, CDCl3): delta 189, 171, 161, 152, 148, 141.9, 129, 123, 92.5, 65.4, 63.9, 49.9, 46.1, 26.2, 25.7, 25.6, 25.4, 18.2, 18.1, −3.00, −3.63, −4.53, −4.74, −5.41, −5.99. IR (KBr, cm−1): 2956, 2931, 2858, 2140, 1746, 1721, 1656, 1525, 1348, 1312, 1255, 840.

### 4-{3-(Imino)-3-[(p-nitrophenyl)methoxycarbonyl]-2-oxopropyl}-2-oxo-3-azetidinecarboxylic Acid

A solution of the silyl enol ether (7.3 g, 12.4 mmol) in DCM (100 mL) was treated with 03 at −78° C. until a blue color persisted. The solution was then purged with a stream of N2 bubbles until it was colorless. To the resulting solution, Me2S (10 mL) was added, and the mixture was stirred at rt for 2 hs and washed with cold water twice. The organic layer was separated and dried over Na2SO4 and concentrated to afford a viscous solid (6.1 g, 81%). 1H NMR (400 MHz, CDCl3): delta 8.29 (d, 2H), 7.57 (d, 2H), 5.39 (s, 2H), 4.21 (dd, 1H), 3.90 (d, 1H), 3.70 (d, 1H), 2.98 (m, 1H), 0.96 (m, 18H), 0.04 (m, 12H). 13C NMR (400 MHz, CDCl3): delta 173, 168, 167, 153, 138, 129, 129, 128.9, 127, 126, 125, 69.8, 60.5, 57.7, 55.3, 38.3, 26.6, 26.1, 25.9, 25.6, 25.5, 25.4, 18.9, 17.8, 15.4, 15.1, −3.63, −4.75, −4.92, −5.16, −5.87, −5.96. IR (KBr, cm−1):2957, 2931, 2860, 2349, 2142, 1754, 1720, 1655, 1608, 1525, 1472, 1381, 1348, 1315, 1257, 1196, 1129, 1001, 948, 842, 809, 742, 696.

### (p-Nitrophenyl)methyl 4-[3-(chloroformyl)-4-oxo-2-azetidinyl]-2-(imino)acetoacetate

A solution of the silyl ester (6.3 g, 10.4 mmol) in DCM (50 mL) was cooled at 0° C. under N2, and DMF (5 drops) was added, followed by oxalyl chloride (3.57 mL, 41.6 mmol). The mixture was stirred at rt for 0.5 h. The resulting yellow solution was evaporated to dryness to afford the titled compound 34 (5.29 g, 99%). 1HNMR (400 MHz, CDCl3): 1H NMR (400 MHz, CDCl3): delta 8.29 (d, 2H), 7.57 (d, 2H), 5.38 (s, 2H), 4.26 (d, 2H), 4.25 (m, 1H), 3.65 (dd, 1H), 2.98 (dd, 1H), 0.88 (s, 9H), 0.05 (d, 6H). 13C NMR (400 MHz, CDCl3): delta 188, 167, 163, 160, 148, 142, 129, 142, 129, 124, 71.6, 65.6, 49.6, 44.6, 25.9, 18.4, 0.96, −5.57, −5.87.

### (p-Nitrophenyl)methyl 4-[3-(hydroxymethyl)-4-oxo-2-azetidinyl]-2-(imino)acetoacetate

To a solution of the acid chloride (5.3 g, 10.4 mmol) in DCM (50 mL), at −78° C. under N2 was added dropwise to a solution of cold tetrabutylammonium borohydride (2.68 g, 10.4 mmol) in DCM (1 mL). The mixture was stirred at −78° C. for 30 min and then the reaction was quenched with TFA (1.60 mL, 4.82 mmol). The organic phase was washed with NH4Cl and brine, dried over Na2SO4, and concentrated to afford a viscous solid. The crude material was purified by flash chromatography (CH3OH: DCM, 0.5% to 20%) to give the titled product (2.09 g, 46%). 1HNMR (400 MHz, CDCl3): delta 8.29 (d, 2H), 7.57 (d, 2H), 5.39 (s, 2H), 3.99 (d, 2H), 3.86 (dd, 1H), 3.60 (dd, 1H), 3.11 (m, 2H), 2.98 (m, 1H), 0.97 (s, 9H), 0.08 (d, 2H). 13C NMR (400 MHz, CDCl3): delta 191, 173, 172, 160, 148, 142, 129, 124, 65.6, 61.2, 60.9, 50.5, 48.8, 26.2, 18.4, 1.03, −5.29, −5.69.

### (p-Nitrophenyl)methyl 4-[3-(hydroxymethyl)-4-oxo-2-azetidinyl]-2-(imino)acetoacetate

Alcohol (14.9 g, 60.32) was dissolved in CH3CN (400 mL) and 15 mL of HF was added to the reaction mixture. Additional amount of HF (20 mL) was added to the reaction to complete. After the reaction completed, a finely ground powder of NaHCO3 was added to the mixture to adjust pH to 8. The solid was filtered and the filtrate was concentrated under reduced pressure to afford a white solid (11 g, 50%).1H NMR (400 MHz, CDCl3): delta 8.22 (d, 2H), 7.55 (d, 2H), 6.90 (s, 1H), 5.25 (s, 2H), 4.10 (m, 3H), 3.06 (m, 2H), 2.90 (s, 1H), 13C NMR (400 MHz, CDCl3): delta 190, 169, 161, 148, 142, 129, 124, 124, 65.6, 59.0, 58.7, 47.5, 44.9.

### p-Nitrophenyl 6-(hydroxymethyl)-3,7-dioxoazabicyclo[3.2.0]heptane-2-carboxylate

(p-Nitrophenyl)methyl 4-[3-(hydroxymethyl)-4-oxo-2-azetidinyl]-2-(imino)acetoacetate (2.7 mg, 7.42 mmol) was dissolved in EtOAc (45 mL). A catalytic amount of Rh(OAc)4 (15 mg) was added to the reaction mixture. The reaction was refluxed to 50° C. for 1 h. The reaction was completed when bubbling subsided. The solvent was removed under reduced pressure to dryness. The entire compound was utilized directly for the next step. The yield was (2.40 g, 98%). 1HNMR (400 MHz, CDCl3): delta 8.27 (d, 2H), 7.55 (d, 2H), 5.32 (dd, 2H), 4.81 (s, 1H), 4.10 (m, 2H), 3.46 (t, 1H), 2.98 (dd, 1H), 2.58 (dd, 1H).

### p-Nitrophenyl 3-{5-(dimethylamino)carbonyl-1-[(p-nitrophenyl)methoxycarbonyl]-3-pyrrolidinylthio}-6-(hydroxymethyl)-7-oxoazabicyclo[3.2.0]hept-2-ene-2-carboxylate

This beta-ketoester (2.58 g, 7.72 mmol) was dissolved in CH3CN and cooled to −40° C. Diphenyl phosphoryl chloride (1.59 mL, 7.72 mmol) was added first and then DIPEA (1.34 ml, 0.738 mmol) was added slowly to the reaction and stirred for 45 min. The reaction was monitored by 1H NMR. Upon completion of the reaction, thiol (2.73 g, 7.72 mmol) and additional amounts of DIPEA (1.34 mL, 7.72 mmol) were added to the reaction and stirred for 1.5 h. Upon completion of the second half of the reaction, the solvent was removed and EtOAc was added to the residue and washed with NaHCO3, NH4Cl, dried over Na2SO4, and the solvent was removed under reduced pressure. The crude material was purified using column chromatography (DCM, MeOH as a gradient eluent from 0-10% MeOH). The yield was (400 mg, 15%). 1HNMR (400 MHz, CDCl3): delta 8.21 (dd, 2H), 7.51 (d, 2H), 7.31 (d, 2H), 7.22 (d, 2H), 5.30 (d, 2H), 5.24 (d, 2H), 4.70 (t, 1H), 4.04 (t, 1H), 3.36 (t, 3H), 3.08 (d, 6H), 2.97 (t, 2H), 2.18 (t, 1H), 1.85 (t, 2H).

### (5R,6S)-6-Hydroxymethyl-3-[[(3S,5S)5-[(dimethylamino)carbonyl-3-pyrrolidinyl]thio]-7-oxo-1-azabicyclo[3.2.0]hept-2-ene-2-carboxylate Acid (1)

A slurry of PNB ester (150 mg, 0.219 mmol) with Pd/C (150 mg) in EtOAc (20 mL) and aq phosphate buffer (pH=6, 20 mL) was shaken on a Parr hydrogenator at 60 psi for 90 min. The solution was then filtered through celite to remove the catalyst. The aqueous layer was separated and washed with ether, then concentrated in vacuo and purified through column chromatography (Diaion CHP/20P).

Yield 19%, 1H NMR (400 MHz, D20): delta 1.79 (q, J=7.56, 1H), 2.89 (m, 1H), 2.98 (2s, 6H), 3.20 (q, J=9.04, 2H), 3.49 (m, 2H), 3.51 (m, 2H), 3.90 (q, J=4.36, 2H), 4.21 (m, 1H), 4.39 (m, 1H).

### Experimental Section, Scheme 2

Methylmagnesium iodide. A dry 500 mL 3-necked round-bottom flask (w/stir bar) with a reflux condenser was charged with magnesium turnings (30.5 g, 1.26 mol, 1.5 eq) in 200 mL of anhyd Et2O and a catalytic amount of iodine crystals were added. A solution of methyl iodide (118.6 g, 52 mL, 0.835 mmol) in 50 mL of anhyd Et2O was then added to the solution dropwise at a rate as to maintain gentle reflux. The reaction was allowed to stir for 3-24 h.

Copper iodide dimethyl sulfide complex. A dry 5 L 3-neck round-bottom-flask (w/overhead stir) was charged with copper iodide (79 g, 0.42 mol) in 2 L of anhyd THF. Dimethyl sulfide (25.8 g, 30.72 mL, 0.42 mol) was added to the solution at rt, and the solution was then cooled to −60° C.

4-Methyl-3-[1-(t-butyldimethylsilyloxy)ethyl]-2-azetidinone. The copper iodide dimethyl sulfide mixture was cooled to −60° C. and methylmagnesium iodide solution was added to the flask. The mixture was warmed to −10 to 0° C. and was allowed to stir for 30 min. The mixture was then cooled to −60° C. and azetidinone (4-Oxo-3-[1-(1,1,2,2-tetramethylpropoxy)ethyl]-2-azetidinyl acetate) (60 g, 0.208 mol) was added to the flask. The reaction was allowed to warm to rt over the course of 45 min. The reaction was quenched with slow addition of satd aq NH4Cl. The mixture was poured into a 3 L round-bottom flask and the THF was removed. The product was dissolved in EtOAc and washed with 2× dilute aq NH4OH solution. The organic layer was dried over Na2SO4 and concentrated in vacuo. The product was purified by silica gel flash chromatography via gradient elution (2.5:97.5 EtOAc/CH2Cl2 to 40/60 EtOAc/CH2Cl2) to afford 3 (50 g, 83% yield) as a white solid. 1H NMR (400 MHz, CDCl3): delta 6.38 (s, 1H), 5.29 (s, 1H), 4.19 (m, 1H), 3.83 (m, 1H), 2.69 (m, 1H), 1.3 (dd, J=54 Hz, 3H), 0.86 (s, 9H), 0.067 (S, 6H). 13C NMR (400 MHz, CDCl3): delta 168.99, 65.74, 65.50, 47.88, 25.74, 25.59, 22.33, 20.59, 17.77, −4.34, −4.62, −4.75.

2-Methyl-4-oxo-3-[1-(t-butyldimethylsilyloxy)ethyl)ethyl]-2-azetidinyl acetate. A dry 100 mL round-bottom flask (w/stir bar) was charged with compound 3 (5 g, 20 mmol) in 70 mL of dry EtOAc. The solution was subsequently treated with sodium acetate (1.5 g, 18.3 mmol, 0.9 eq), acetic acid (11.55 g, 11 mL, 192 mmol, 9.6 eq) and ruthenium trichloride (dried using a Bunsen burner under vacuum) (0.3 g, 1.44 mmol, 7 mol %). The flask was cooled to 12-15° C. and oxygen pressure (12 psi) was applied to the reaction. Acetaldehyde (11.8 g, 15 mL, 268 mmol, 13.4 eq) was freshly distilled twice and added via syringe. The reaction was monitored by NMR. Once completed, the reaction was poured into cold hexane (1 L). The mixture was extracted with cold hexane and washed with cold satd aq NaCl until the pH reached 7. The organic layer was dried over Na2SO4 and evaporated in vacuo to afford 4 (4.55 g, 91% yield) as a purple oil form. 1H NMR (400 MHz, CDCl3): delta 7.05 (S, 1H), 4.31 (m, 1H), 3.05 (d, J=9.2 Hz, 1H), 2.04 (s, 3H), 1.82 (s, 3H), 1.33 (d, 6 Hz, 3H), 0.86 (s, 9H), 0.067 (s, 6H). 13C NMR (400 MHz, CDCl3): delta 170.22, 166.39, 88.61, 70.25, 68.75, 67.32, 64.69, 25.49, 21.90, 19.69, 17.64, 0.82, −3.98, −4.41.

(p-Nitrophenyl)methyl 2-diazo-3-(t-butyldimethylsilyloxy)-3-butenoate. A dry 1 L round-bottom flask (w/stir bar) was charged with p-nitrobenzyl-2-diazoacetoacetate (52.6 g, 0.2 mol) in 400 mL dry CH2Cl2, and the flask was cooled to 0° C. Triethylamine (40 mL, 0.29 mol, 1.4 eq) was added to the flask and TBS-OTf (63.44 g, 55 ml, 0.24 mol, 1.2 eq) was added. The reaction was warmed to room temperate and allowed to stir until completed. Once completed, the solution was washed three times with ice water. The organic layer was dried over Na2SO4, evaporated in vacuo and further dried under high vacuum overnight. 1H NMR (400 MHz, CDCl3): delta 7.92 (dd, J=287 Hz, 8.4 Hz 4H), 5.31 (s, 2H), 4.98 (s, 1H), 4.98 (s, 1H), 0.86 (s, 9H), 0.07 (s, 6H). NMR (400 MHz, CDCl3): delta 163.64, 160.90, 147.91, 147.69, 143.04, 142.18, 140.29, 128.62, 128.28, 123.96, 123.82, 90.56, 65.34, 64.73, 28.25, 25.60, 25.52, 18.04, 17.93, −3.64, −4.84. (p-Nitrophenyl)methyl2-diazo-4-{2-methyl-4-oxo-3-[1-(t-butyldimethylsilyloxy)ethyl]-2-azetidinyl}acetoacetate. A dry 100 mL round-bottom flask (w/ stir bar) with a reflux condenser was charged with acetate (3 g, 10.4 mmol) and TBS enol ether (5.9 g, 15.6 mmol, 1.5 eq) in 25 mL dry CH2Cl2. 1M ZnCl2 in ether (7.3 mL, 7.28 mmol, 0.7 eq) was added to the reaction and the flask was heated to 42° C. for 20 min at reflux. Once completed, the reaction was cooled to rt and then diluted with EtOAc. The solution was washed with sated aq NaHCO₃ once and extracted with EtOAc twice. The organic layer was dried over Na2SO4 and evaporated in vacuo. The crude material was purified by silica gel flash chromatography via gradient elution (2.5:97.5 EtOAc/CH2Cl2 to 40/60 EtOAc/CH2Cl2) to afford product (0.92 g, 31% yield) as solid. 1H NMR (400 MHz, CDCl3): delta 7.92 (dd, J=287 Hz, 8.4 Hz 4H), 6.38 (s, 1H), 5.35 (d, J=14 Hz, 2H), 4.27 (t, J=2.8 Hz, 1H), 3.59 (dd, J=292 Hz, 16.8 Hz, 2H), 2.85 (s, 1H), 1.538 (s, 3H), 1.40 (d, J=12, 3H), 1.33 (t, J=13.6 Hz, 2H), 0.085 (s, 9H), 0.866 (S, 6H). 13C NMR (400 MHz, CDCl3): 189.84, 167.09, 160.56, 147.85, 141.95, 128.66, 128.56, 123.88, 123.81, 66.80, 65.43, 65.21, 55.71, 49.90, 25.69, 25.33, 22.29, 21.15, 19.78, 17.76, −3.30, −4.82.

(p-Nitrophenyl)methyl2-diazo-4-[3-(1-hydroxyethyl)-2-methyl-4-oxo-2-azetidinyl]acetoacetate. A 50 mL round-bottom flask (w/ stir bar) was charged with (p-nitrophenyl)methyl2-diazo-4-{2-methyl-4-oxo-3-[1-(t-butyldimethylsilyloxy)ethyl]-2-azetidinyl}acetoacetate (2 g, 7.8 mmol) in 20 mL of dry acetonitrile, and 1 mL of HF was added. The reaction was stirred at rt. Once completed (1-3 h), finely ground NaHCO₃ was added to the reaction to attain pH=7. The reaction was filtered to remove the NaF and evaporated in vacuo to afford product (1.4 g, 71% yield) as white solid. 1H NMR (400 MHz, CDCl3): delta 7.91 (dd, J=280 Hz, 8.3 Hz, 4H), 5.34 (q, J=18.4 Hz, 13.2 Hz, 2H), 4.84 (s, 1H), 4.39 (m, 1H), 3.22 (d, J=10′ Hz, 1H), 2.7 (dd, J=44.4, 18 Hz, 2H), 1.63 (s, 3H), 1.42 (d, 6.4 Hz, 3H), 0.89 (s, 1H), 0.085 (s, 2H). 13C NMR (400 MHz, CDCl3): delta 190.53, 167.18, 160.14, 147.43, 141.86, 128.37, 123.50, 65.32, 63.51, 54.89, 49.67, 49.07, 48.64, 48.00 21.24, 20.29.

(p-Nitrophenyl)methyl6-(1-hydroxyethyl)-5-methyl-3,7-dioxoazabicyclo[3.2.0]heptane-2-carboxylate. A 100 mL round-bottom flask (w/ stir bar) with a reflux condenser was charged with (p-nitrophenyl)methyl2-diazo-4-[3-(1-hydroxyethyl)-2-methyl-4-oxo-2-azetidinyl]acetoacetate (1.3 g, 3.33 mmol) and Rh2(OAc)4 (35 mg, 0.08 mm, 0.024 eq) in 50 mL dry EtOAc. The reaction was heated to 60° C. for 30 min. Once completed, reaction was cooled to room temperature and was evaporated in vacuo. 1H NMR (400 MHz, CDCl3): delta 7.96 (dd, J=232 Hz, 8.4 Hz, 4H), 5.31 (q, J=19.6 2H), 4.15 (q, 8 Hz, 1H), 3.68 (q, 10 Hz, 1H), 3.18 (d, 4 Hz, 1H), 2.65 (dd, J=40 Hz, 20 Hz, 1H)), 2.08 (s, 3H), 1.55 (dd, J=30 Hz, 15 Hz, 3H), 1.45 (d, 15 Hz, 1H), 1.28 (m, 1H).

(p-Nitrophenyl)methyl6(S)-6-[(R)-1-hydroxyethyl]-3-{(3S,5S)-5-(dimethylamino)carbonyl-1-[(p-nitrophenyl)methyl]-3-pyrrolidinylthio}-5-methyl-7-oxoazabicyclo[3.2.0]hept-2-ene-2-carboxylate. A 50 mL round-bottom flask (w/stir bar) was charged with (p-nitrophenyl)methyl6-(1-hydroxyethyl)-5-methyl-3,7-dioxoazabicyclo[3.2.0]heptane-2-carboxylate (1.3 g, 3.59 mmol) in 10 mL of dry CH3CN and was cooled to −35° C. Diphenyl phosphoryl chloride (0.93 g, 0.72 mL, 3.59 mmol, 1 eq) was then added to the flask followed by a slow addition of N,N-diisopropylethylamine (0.45 g, 3.5 mmol, 1 eq), and the reaction was allowed to stir for 30 minutes. Once completed, 4-nitrobenzyl (2S,4S)-2-(dimethylcarbamoyl)-4-mercapto-1-pyrrolidinecarboxylate (1.26 g, 0.9 mL, 3.59 mmol, 1 eq) and an additional 1 eq of DIPEA was added. Once completed, the reaction was extracted with CH2Cl2 and washed with satd aq NH4Cl. The resultant solution was then evaporated in vacuo to afford product (1.1 g, 85% yield) as solid. 1H NMR (400 MHz, CDCl3): delta 8.25 (d, 8.8 Hz, 2H), 7.67 (d, 8.4 Hz, 2H), 7.50 (dd, 28.8 Hz, 8.4 Hz, 2H), 5.54 (d, 13.6 Hz, 2H), 5.24 (m, 1H), 4.74, (m, 1H), 4.31 (m, 1H), 3.57 (m, 1H), 3.25 (dd, 56 Hz, 14.4 Hz, 2H), 3.09 (dd, 78 Hz, 4 Hz, 6H), 2.8 (m, 1H), 1.99 (m, 1H), 1.62 (d, 6 Hz, 3H), 1.45 (d, 6 Hz, 3H).

(6S)-6-[(R)-1-Hydroxyethyl]-3-[(3S,5S)-5-(dimethylamino)carbonyl-3-pyrrolidinylthio]-5-methyl-7-oxoazabicyclo[3.2.0]hept-2-ene-2-carboxylate (2a). A 100 mL round-bottom flask (w/ stir bar) was charged with (p-nitrophenyl)methyl6(S)-6-[(R)-1-hydroxyethyl]-3-{(3S,5S)-5-(dimethylamino)carbonyl-1-[(p-nitrophenyl)methyl]-3-pyrrolidinylthio}-5-methyl-7-oxoazabicyclo[3.2.0]hept-2-ene-2-carboxylate (0.55 g, 0.79 mmol) in 80 mL of dry EtOAc and 40 mL of pH6 phosphate buffer solution (0.3 M). After dissolved, 10% Pd on carbon (0.55 g, 5.2 mmol, 6.6 eq) was added, and the vessel was subjected to hydrogen pressure 55 psi in a Parr hydrogenation device for 90 min. Once completed, the solution was filtered through celite, the aqueous layer was separated and washed with Et2O. The organic solvents were then removed from the aqueous layer and the product isolated by column chromatography on Diaion CHP20P resin. Tubes containing the product were identified by inspection of the UV of each fraction, combined, and the water was partially removed in vacuo. The remaining aqueous solution was lyophilized to produce the purified antibiotic 2a (0.1385 g, 25% yield) as white solid. 1H NMR (400 MHz, CDCl3): delta 4.85 (s, 1H), 4.60 (t, 8.4 Hz, 1H), 4.33 (m, 1H), 3.98 (t, 6.4 Hz, 1H), 3.64 (q, 6.8 Hz, 1H), 3.36 (m, 2H), 3.09 (d, 47 Hz, 6H), 2.90 (d, 22 Hz, 2H), 1.87 (m, 1H), 1.55 (s, 3H), 1.34 (d, 66 Hz, 3H). 13C NMR (400 MHz, CDCl3): delta 178.0, 170.0, 168.5, 137.5, 129.7, 66.5, 66.4, 63.00, 60.5, 58.8, 52.0, 47.6, 41.7, 37.5, 36.5, 36.4, 22.0, 21.5,

Ethylmagnesium iodide. A dry 500 mL-3-neck round bottom flask. (w/ stir bar) with a reflux condenser was charged with magnesium turnings (30.5 g, 1.26 mol, 1.5 eq) and a catalytic amount of iodine crystals in 200 mL of anhyd Et2O. A solution of ethyl iodide (130 g, 67 mL, 0.835 mol) in Et2O was added to the solution dropwise at a rate to maintain gentle reflux.

(3S)-3-[(R)-1-(t-butyldimethylsilyloxy)ethyl]-4-ethyl-2-azetidinone. The copper iodide dimethyl sulfide (2) mixture was cooled to −60° C. and ethylmagnesium iodide was added to the flask. The mixture was warmed to −10° C. and was allowed to stir for 5 minutes. The flask was cooled to −60° C. and acetate Azetidinone (60 g, 0.209 mmol) was added to the flask. Once the reaction completed, it was warmed to room temperature. The reaction was quenched with slow addition of satd aq NH4Cl. The mixture was poured into a 3 L round-bottom flask and evaporated in vacuo. The product was dissolved in EtOAc and washed with dilute NH4OH. The EtOAc layer was dried over Na2SO4 and evaporated. The product was then purified by silica gel flash chromatography via gradient elution (2.5:97.5 EtOAc/CH2Cl2 to 40/60 EtOAc/CH2Cl2) to afford 15 (48.7 g, 81% yield) as a white solid. 1H NMR (400 MHz, CDCl3): delta 5.32 (s, 1H), 2.5 (m, 1H), 2.14 (s, 1H), 1.87 (t, 3.2 Hz, 3H), 1.26 (q, 2.4 Hz, 2H), 0.92 (s, 9H), 0.094 (s, 6H).

(3R)-3-[(R)-1-(t-butyldimethylsilyloxy)ethyl]-2-ethyl-4-oxo-2-azetidinyl acetate. A dry 500 mL round-bottom flask (w/ stir bar) was charged with (3S)-3-[(R)-1-(t-butyldimethylsilyloxy)ethyl]-4-ethyl-2-azetidinone (10 g, 38.8 mmol) in 280 mL of dry EtOAc. The solution was subsequently treated with sodium acetate (1.8 g, 22.0 mmol, 0.6 eq), acetic acid (23.1 g, 22 mL, 384 mmol, 9.9 eq) and ruthenium trichloride (dried using a Bunsen burner under vacuum) (3 g, 14 mmol, 37 mol %) was added to the flask. The flask was then cooled to 12-15° C. and oxygen pressure (12 psi) was applied to the reaction. Acetaldehyde (23.6 g, 30 mL, 536 mmol, 13.8 eq) was freshly distilled twice and added via syringe. The flask was kept closed at all times. Once completed, the reaction was poured into cold hexane. The mixture was extracted with cold hexane and washed with iced satd aq NaCl until pH reached 7. The organic layer was dried over Na2SO4 and evaporated in vacuo to afford product (4.55 g, 91% yield) as purple oil form. 1H NMR (400 MHz, CDCl3): delta 5.89 (s, 1H), 4.29 (m, 1H), 2.64 (q, J=0.8 Hz, 2H), 1.27 (d, 3 Hz, 3H), 1.05 (t, J=3.2 Hz, 3H), 0.873 (s, 9H), 0.088 (s, 6H). 13C NMR (400 MHz, CDCl3): 170.38, 166.22, 90.01, 66.10, 64.47, 28.77, 25.51, 22.21, 21.52, 17.72, 9.02, 0.84, −4.13, −4.76.

(p-Nitrophenyl)methyl2-diazo-4-{2-ethyl-4-oxo-3-[1-(t-butyldimethylsilyloxy)ethyl]-2-azetidinyl}acetoacetate. A dry 250 mL round-bottom flask (w/stir bar) with a reflux condenser was charged with (3R)-3-[(R)-1-(t-butyldimethylsilyloxy)ethyl]-2-ethyl-4-oxo-2-azetidinyl acetate (10 g, 38.85 mmol) and (p-nitrophenyl)methyl 2-diazo-3-(t-butyldimethylsilyloxy)-3-butenoate (21.2 g., 56.5 mmol, 1.5 eq) in 50 mL dry CH2Cl2. 1M ZnCl2 in Et2O (27 mL, 27.7 mmol, 0.7 eq) was added to the reaction and the flask was heated to 48° C. Once completed, the reaction was cooled to room temperature and then diluted with EtOAc. The solution was washed with satd aq NaHCO₃ once and extracted with EtOAc twice. The organic layer was dried over Na2SO4 and then evaporated in vacuo. The crude material was purified by silica gel flash chromatography via gradient elution (2.5:97.5 EtOAc/CH2Cl2 to 40/60 EtOAc/CH2Cl2) to afford product (3.9 g, 39% yield) as solid. 1H NMR (400 MHz, CDCl3): delta 7.93 (d, J=282 Hz, 20.4 Hz, 4H), 6.21 (s, 1H), 5.36 (d, J=36.8 Hz, 2H), 4.29 (m, 1H), 3.74 (dd, 343 Hz, 14.8 Hz, 2H), 3.14 (d, 8.8 Hz, 1H), 2.02 (q, 7.6 Hz, 1H), 1.90 (q, 7.6 Hz, 1H), 1.34 (d, 36.8 Hz, 3H), 1.26 (t, 7.2 Hz, 3H), 0.932 (s, 9H), 0.138 (s, 6H). 13C NMR (400 MHz, CDCl3): delta 199.20, 189.52, 168.88, 167.61, 160.90, 147.94, 141.85, 128.63, 123.95, 65.73, 61.61, 59.41, 52.50, 46.92, 27.89, 25.69, 22.64, 22.68, 17.82, 10.60, 8.05, 0.92, −3.33, −4.36.

(p-Nitrophenyl)methyl2-diazo-4-[3-(1-hydroxyethyl)-2-ethyl-4-oxo-2-azetidinyl]acetoacetate. A 100 mL round-bottom flask (w/stir bar) was charged with (p-nitrophenyl)methyl2-diazo-4-{2-ethyl-4-oxo-3-[1-(t-butyldimethylsilyloxy)ethyl]-2-azetidinyl}acetoacetate (6 g, 7.8 mmol) in 20 mL of acetonitrile, and 1 mL of HF was then added. The reaction stirred at rt and monitored by TLC. Once completed, finely ground NaHCO₃ was added to the reaction to retain pH=7. The reaction was filtered to remove the NaF and evaporated in vacuo to afford product (2.34 g, 39% yield) as white solid. 1H NMR (400 MHz, CDCl3): delta 7.90 (dd, 289 Hz, 8.5 Hz, 4H), 5.35 (s, 2H), 4.23 (m, 1H), 3.35 (dd, 260 Hz, 18.4 Hz, 2H), 2.05 (m, 1H), 1.88 (m, 1H), 1.35, (d, J=4 Hz, 3H), 0.92 (t, J=7 Hz, 3H). 13C NMR (400 MHz, CDCl3): delta 190.98, 167.27, 160.44, 147.99, 141.88, 128.82, 124.02, 66.76, 65.74, 63.57, 58.61, 46.13, 26.04, 21.85, 8.57.

(p-Nitrophenyl)methyl5-ethyl-6-(1-hydroxyethyl)-3,7-dioxoazabicyclo[3.2.0]heptane-2-carboxylate. A dry 100 mL round-bottom flask (w/ stir bar) with a reflux condenser was charged with (p-nitrophenyl)methyl2-diazo-4-[3-(1-hydroxyethyl)-2-ethyl-4-oxo-2-azetidinyl]acetoacetate (2.34 g, 5.79 mmol) and Rh2(OAC)4 (50 mg, 0.11 mmol, 0.019 eq) in 40 mL dry EtOAc. The reaction was heated to 80° C. for 30 min. Once completed, reaction was cooled to room temperature and was evaporated in vacuo. 1H NMR (400 MHz, CDCl3): delta 7.94 (272 Hz, 88 Hz, 4H), 5.40 (38 Hz, 13.2 Hz, 2H), 4.83 (s, 1H), 4.45 (m, 1H), 3.25 (d, 9.6 Hz, 1H), 2.5 (158 Hz, 17.6 Hz, 2H), 1.95 (m, 2H), 1.49 (d, 3.5 Hz, 3H), 1.16 (108 HZ, 7.2 Hz, 3H). 13C NMR (400 MHz, CDCl3): delta 189.62, 167.71, 161.00, 141.95, 128.73, 124.05, 65.55, 61.71, 52.60, 46.02, 27.46, 25.75, 22.68, 17.92, 10.70, 8.15, 1.02, −3.23, −4.26.

(p-Nitrophenyl)methyl (6S)-6-[(R)-1-hydroxyethyl]-3-{(3S,5S)-5-(dimethylamino)carbonyl-1-[(p-nitrophenyl)methyl]-3-pyrrolidinylthio}-5-ethyl-7-oxoazabicyclo[3.2.0]hept-2-ene-2-carboxylate (20). A dry 50 mL round-bottom flask (w/ stir bar) was charged with (p-nitrophenyl)methyl5-ethyl-6-(1-hydroxyethyl)-3,7-dioxoazabicyclo[3.2.0]heptane-2-carboxylate (2.34 g, 6.22 mmol) in 30 mL of dry CH3CN and was cooled to −35° C. Diphenyl phosphoryl chloride (1.4 g, 1.1 mL, 6.22 mmol, 1 eq) was added to the flask followed by a slow addition of N,N-diisopropylethylamine (0.74 g, 1 mL, 6.22 mmol, 1 eq). Once completed, the side chain, 4-nitrobenzyl (2S,4S)-2-(dimethylcarbamoyl)-4-mercapto-1-pyrrolidinecarboxylate (1.26 g, 0.9 mL, 3.59 mmol, 1 eq) and an additional 1 eq of DIPEA was added. Once completed, the reaction was extracted with CH2Cl2 and washed with satd aq NH4Cl. The resultant solution was then evaporated in vacuo to afford product (1.8 g, 77% yield) as solid. 1H NMR (400 MHz, CDCl3): delta 8.20 (m, 4H), 7.64 (d, J=8.8 Hz, 2H), 7.47 (dd, J=35 Hz, 8.8 Hz, 2H), 5.52 (d, J=14 Hz, 2H), 5.22 (m, 1H), 5.18 (dd, J=77.2 Hz, 14 Hz, 2H), 4.15 (m, 1H), 3.65 (m, 1H), 3.55 (m, 1H), 3.26 (m, 2H), 3.09 (d, J=78 Hz, 6H), 2.75 (m, 1H), 2.04 (m, 1H), 1.91 (m, 2H), 1.40 (d, J=6 Hz, 3H), 0.99 (t, J=7.2 Hz, 3H). 13C NMR (400 MHz, CDCl3): delta 175.86, 170.52, 160.54, 153.49, 153.02, 147.43, 146.01, 143.75, 143.06, 127.95, 124.34, 124.21, 123.66, 123.57, 69.04, 65.73, 65.13, 64.28, 64.17, 56.18, 55.85, 53.80, 52.90, 45.98, 41.44, 40.70, 37.14, 36.89, 36.06, 27.60, 22.67, 7.68, 0.91.

(6S)-6-[(R)-1-Hydroxyethyl]-3-[(3S,5S)-5-(dimethylamino)carbonyl-3-pyrrolidinylthio]-5-ethyl-7-oxoazabicyclo[3.2.0]hept-2-ene-2-carboxylate (2b). A parr hydrogenation vessel was charged with (p-nitrophenyl)methyl (6S)-6-[(R)-1-hydroxyethyl]-3-{(3S,5S)-5-(dimethylamino)carbonyl-1-[(p-nitrophenyl)methyl]-3-pyrrolidinylthio}-5-ethyl-7-oxoazabicyclo[3.2.0]hept-2-ene-2-carboxylate (0.6 g, 0.86 mmol) in 30 mL of EtOAc and 30 mL of pH6 NaH2PO4 buffer solution (0.3 M). After dissolved, 10% Pd on carbon (0.6 g, 5.6 mmol, 6.5 eq) was added, and the vessel was subjected to hydrogen pressure 55 psi in a Parr hydrogenation device for 90 min. Once completed, the solution was filtered, the aqueous layer was separated and washed with Et2O. The organic solvents were then removed from the aqueous layer and the product isolated by column chromatography on Diaion CHP20P resin. Tubes containing the product were identified by inspection of the UV of each fraction, combined, and the water was partially removed in vacuo. The remaining aqueous solution was lyophilized to produce the purified antibiotic 2b (0.15 g, 25% yield) as white solid. 1H NMR (400 MHz, CDCl3): delta 4.58 (s, 1H), 3.43 (m, 2H), 3.29 (d, 17.6 Hz, 2H), 3.02 (d, 4.4 Hz, 6H), 1.98 (m, 1H), 1.85 (m, 1H), 1.33 (d, 6.4 Hz, 3H), 0.97 (t, 7.2 Hz, 3H). 13C NMR (400 MHz, CDCl3): delta 182.09, 171.06, 170.79, 138.38, 134.87, 67.02, 66.44, 61.01, 54.74, 47.27, 43.88, 39.31, 38.53, 38.05, 29.90, 214.12, 18.83, 9.83.

TABLE 1
MICs (mg/L) of carbapenems against resistant Acinetobacter baumannii,
relative to commercial carbapenem antibiotics

| | Carbapenemase Produced | | |
|---|---|---|---|
| Compound | None | OXA-23 | OXA-48 |
| 1 | 0.25 | 8 | 16 |
| 2a | 0.5 | 4 | 1 |
| 2b | 2 | 4 | 4 |
| Meropenem | 0.5 | 64 | 8 |
| Doripenem | 0.25 | 32 | 16 |
| Ertapenem | 2 | 256 | 128 |
| Imipenem | 0.25 | 32 | 16 |

TABLE 2
MICs (mg/L) of compound 2b, relative to commercial antibiotics.
against resistant Acinetobacter baumannii producing
carbapenemases as shown

| Enzyme | | | | | | |
|---|---|---|---|---|---|---|
| produced | AMP | IMP | DOR | ETP | MEM | 2b |
| None | 32 | 0.25 | 0.25 | 4 | 0.5 | 2 |
| OXA-23 | 8192 | 32 | 32 | 256 | 64 | 4 |
| OXA-48 | 1024 | 16 | 16 | 128 | 16 | 4 |
| KPC-6 | 4096 | 32 | 128 | >512 | 256 | 4 |
| NDM-1 | 1024 | 16 | 64 | 256 | 128 | 2 |
| VIM-2 | >8192 | 32 | 32 | >512 | 64 | 4 |

Abbreviations: AMP, ampicillin; IMP, imipenem; DOR, doripenem; ETP, ertapenem; MEM, meropenem

What is claimed:

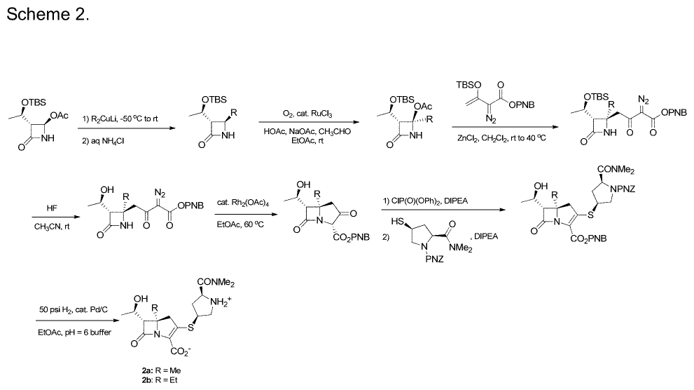

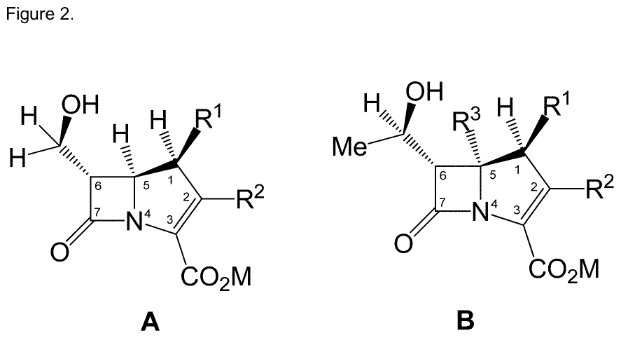

1. Compounds of formulas A and B, as shown in FIG. 2, or a pharmaceutically acceptable salt thereof where:
FIG. 2.
Wherein:
Where R¹═H or CH₃
Where R² may be SR^{a}, where R^{a}=may be an unsubstituted C1 to C6 alkyl group, or substituted C1 to C6 alkyl group, especially including substituents which themselves possess a basic nitrogen, and hence a positive charge. Or alternatively R^{a} may be a substituted or unsubstituted, cyclic or heterocylic group, especially including groups which contain 1 to 3 positive charges, an aryl or heteroaryl group, or substituted aryl or heteroaryl group, particularly including a substituted pyrrolidine.
Where R³ may be Methyl or Ethyl, as seen for compounds 2a and 2b, respectively.
With respect to —CO₂M, which is attached to the carbapenem nucleus at position 3, this represents a carboxylic acid group (M represents H), a carboxylate anion (M represents a negative charge), a pharmaceutically acceptable ester (M represents an ester forming group) or a carboxylic acid protected by a 30 protecting group (M represents a carboxyl protecting group).
The pharmaceutically acceptable salts referred to above may take the form —COOM, where M is a negative charge, which is balanced by a counterion, e.g., an alkali metal cation such as sodium or potassium. Other pharmaceutically acceptable counterions may be calcium, magnesium, zinc, ammonium, or alkylammonium cations such as tetramethylammonium, tetrabutylammonium, choline, triethylhydroammonium, meglumine, triethanolhydroammonium, etc.
The pharmaceutically acceptable salts referred to above also include acid addition salts. Thus, the Formula I compounds can be used in the form of salts derived from inorganic or organic acids. Included among such salts are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate and undecanoate.
The pharmaceutically acceptable esters are such as would be readily apparent to a medicinal chemist, and include, for example, those described in detail in U.S. Pat. No. 4,309,438. Included within such pharmaceutically acceptable esters are those which are hydrolyzed under physiological conditions, such as pivaloyloxymethyl, acetoxymethyl, phthalidyl, indanyl and methoxymethyl, and others described in detail in U.S. Pat. No. 25 4,479,947. These are also referred to as “biolabile esters”.
Biolabile esters are biologically hydrolizable, and may be suitable for oral administration, due to good absorption through the stomach or intestinal mucosa, resistance to gastric acid degradation and other factors. Examples of biolabile esters include compounds in which M represents an alkoxyalkyl, alkylcarbonyloxyalkyl, alkoxycarbonyloxyalkyl, cycloalkoxyalkyl, alkenyloxyalkyl, aryloxyalkyl, alkoxyaryl, alkylthioalkyl, cycloalkylthioalkyl, alkenylthioalkyl, arylthioalkyl or alkylthioaryl group. These groups can be substituted in the alkyl or aryl portions thereof with acyl or halo groups. The following M species are examples of biolabile ester forming moieties.: acetoxymethyl, 1-acetoxyethyl, 1-acetoxypropyl, pivaloyloxymethyl, 1-isopropyloxycarbonyloxyethyl, 1-cyclohexyloxycarbonyloxyethyl, phthalidyl and (2-oxo-5-methyl-1,3-dioxolen-4-yl)methyl.
